(19) United States
(12) Patent Application Publication
(10) Pub. No.: US 2025/0222304 A1
(43) Pub. Date: Jul. 10, 2025

(54) VIBRATION ENHANCED INTERMITTENT HYPOXIA FOR CARDIOVASULAR IMPROVEMENT

(71) Applicant: CoFactor Systems, Inc., Santa Barbara, CA (US)

(72) Inventor: John Foster, Santa Barbara, CA (US)

(73) Assignee: CoFactor Systems, Inc., Santa Barbara, CA (US)

(21) Appl. No.: 18/408,480

(22) Filed: Jan. 9, 2024

(51) Int. Cl. A63B 23/18; A61F 9/04; A61H 23/02; A63B 71/06

(52) U.S. Cl. CPC A63B 23/185; A61F 9/04; A61H 23/02; A61H 2230/208; A61H 2230/30; A63B 2071/063

(57) ABSTRACT

A novel and effective system is described, for substantially altering one's arterial stiffness. The system combines the use of a commercially available, microprocessor-controlled chair with multiple transducers producing vibrations from the torso to the calves, coupled to an oxygen saturation sensor, and a controller which instructs the user on when and how to breathe. The instruction can include hyperventilation, exhaling and holding out one's breath, taking one breath, and other suggestions for the user, as well as providing information such as SpO2 level, heart rate, heart rate variability and other physiological parameters. When the protocol provided herein is used, together the vibration unit, the SpO2 sensor and the controller cause the significant reduction in the user's arterial stiffness over a time on the order of a month.

### CROSS REFERENCE TO RELATED APPLICATIONS

Not applicable.

### STATEMENT REGARDING FEDERALLY SPONSORED RESEARCH

Not applicable.

### STATEMENT REGARDING MICROFICHE APPENDIX

Not applicable.

### BACKGROUND

Breathing techniques, including special breathing and holding one's breath, have long been thought to improve certain health conditions, notably cardiovascular function. In particular, hyperventilation followed by holding one's breath out (air exhausted from lungs) is a standard yoga practice dating 1500-2000 years ago. In addition, a related practice is performed by free-divers, and studies have been performed to show small cardiovascular and other improvements, in particular improvements in large artery elasticity.

### SUMMARY

A system is described which improved cardiovascular fitness by repeatedly inducing a state of intermittent hypoxia in the user. To achieve this state, the user breathes according to a pattern and tempo as instructed by a controller issuing audible or visual instructions (for example, a vocal issuance to breathe or an optical display indicating the same). The breathing pattern is accompanied by applying a vibration to the body of the user. In one embodiment, the vibration is applied to the torso, thorax or ribcage of the user. The effect of the vibration is to alter the oxygen saturation profile, which is reported to the user and to alter the accompanying nitric oxide levels, which can act to remodel the vasculature and the arterial stiffness in particular.

Herein we provide a novel and effective system for substantially altering one's arterial stiffness. The system could combine the use of a commercially available, microprocessor-controlled chair with multiple (18) transducers (www.shiftwave.co) producing vibrations from the torso to the calves, coupled to a blood oxygen saturation sensor, and a controller which instructs the user on when and how to breathe. The instruction can be audible or visual from a messaging device. The instruction can include hyperventilation, exhaling and holding out one's breath, continuing to hold out one's breath, repeatedly taking one breath and holding one's breath out multiple times, breathing for safety reasons, and other suggestions for the user, as well as providing information such as blood saturated oxygen level (SpO2) level, heart rate, heart rate variability, blood pressure, and other physiological parameters. The described protocol produces a reduction in the blood oxygen saturation sensor level of the user, typically by at least 4 percent. When the protocol provided herein is used, on the order of 1 hour per day, together the vibration unit, the SpO2 sensor and the controller cause significant reduction in the user's arterial stiffness over a time on the order of a month.

Accordingly, a therapeutic system for controlling breath of a user is described. The system may include a controller, a vibrating component, an SpO2 sensor, a display, and an audio source, wherein the controller directs the vibration component to create a vibration and apply it to the user, while instructing the user through the audio source to hyperventilate and to exhale the breath and hold the breath out.

A method is also described. The method may include providing a controller coupled to a vibration device, a massaging device and an oxygen saturation sensor, controlling the vibration device with the controller to create a vibration and apply it to the body of the user. instructing the user to exhale the breath and hold the breath out, reporting the oxygen saturation level to the user through the messaging device; and repeating these steps until a predefined oxygen saturation level is achieved.

### BRIEF DESCRIPTION OF THE DRAWINGS

FIG. 1 is a simplified schematic diagram of an embodiment of the therapeutic system;

FIG. 2a shows an exemplary time-dependent profile of the oxygen saturation level of the user in the therapeutic system; FIG. 2b shows the time-dependent vibration amplitude applied to the user and the associated breathing profile of the user in the therapeutic system;

FIG. 3 shows an exemplary time-dependent vibration amplitude, breathing profile, and blood oxygen saturation which occurs over time;

FIG. 4 shows the behavior of the oxygen saturation profile resulting from the breathing profile under several conditions;

FIG. 5 shows exemplary data showing the corresponding apparent age based on the arterial age index;

FIG. 6 shows exemplary data showing the improvement in aortic stiffness; and

FIG. 7 shows exemplary data showing the extended intermittent hypoxia associated with a particular breathing pattern; and

FIG. 8 shows exemplary data showing the number of breaths a user takes after holding breath out until the blood oxygen saturation goes above 97% as a function of the hyperventilation/intermittent hypoxia cycles.

### DETAILED DESCRIPTION

Artery and arteriole elasticity (inversely proportional to stiffness) is an important property of cardiovascular function. As one ages (from approximately age 20), it is known the arteries become less elastic (more stiff). This stiffening is typically the dominant effect of aging on cardiovascular performance, and is linked to heart disease and a host of other conditions including dementia and Alzheimer's Disease. While exercise, eating properly, yoga-type breathing techniques, free-diving and other acts have been shown to provide a decrease in the rate of decline with age, or even modest improvement, these effects are small.

Herein we provide a novel and effective method for substantially altering one's arterial stiffness. As an example, the subject used for the data enclosed has shown a decline of effective arterial age from 55 years old to 26 years old, as a result of using a novel protocol of intermittent hypoxia enhanced with vibration applied to the body. (The subject's actual age is 65.) This improvement is radically larger than has been demonstrated in scientific literature and is an unexpected outcome of the novel protocol herein.

The invention combines the use of a commercially available, microprocessor-controlled chair with multiple transducers producing vibrations from the torso to the calves, coupled to a blood oxygen saturation sensor, and a controller which instructs the user on when and how to breathe. The instruction can include hyperventilation, exhaling and holding out one's breath, taking one breath and exhaling and holding out one's breath, and other suggestions for the user, as well as providing information such as SpO2 level, heart rate, heart rate variability and other physiological parameters. When the protocol provided herein is used, together the vibration unit, the SpO2 sensor and the controller cause the significant reduction in the user's arterial stiffness over a time on the order of a month.

Others have considered the benefits of measuring blood oxygen saturation levels and communicating to the user with intermittent hypoxic and hyperventilating messaging, for example EP 2 934 707 B1, AUTOMATED SYSTEMS, METHODS, AND APPARATUS FOR BREATH TRAINING. However, while this invention may have some benefit, the present invention far surpasses any expected benefit in known literature and must be considered a dramatic and novel improvement.

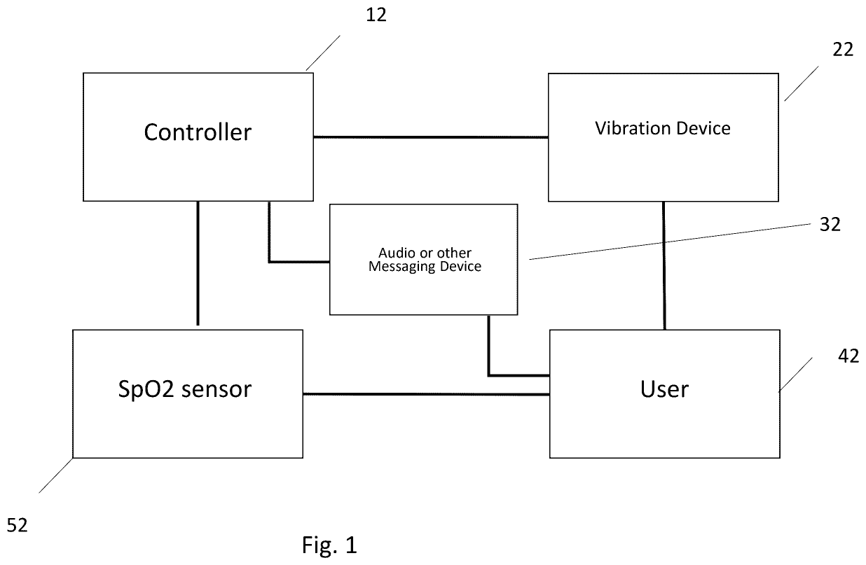

FIG. 1 shows a controller which controls the vibration device (which could be the Shiftwave® chair), which in turn is coupled to the user mechanically. Shiftwave® is the registered trademark of CoFactor Systems of Santa Barbara, Ca. Shown in FIG. 1 are the components controller 12, vibration device 22, and messaging device 32, such as an audio messaging device like a speaker or microphone. Further components may include an oxygen saturation transducer SpO2 52, and a user 42. The controller may be any programmable microprocessor device capable of following software instructions to produce the sensations described below. These sensations may include a “trip” or “narrative”, or a story delivered to the user 42 by the messaging device 32.

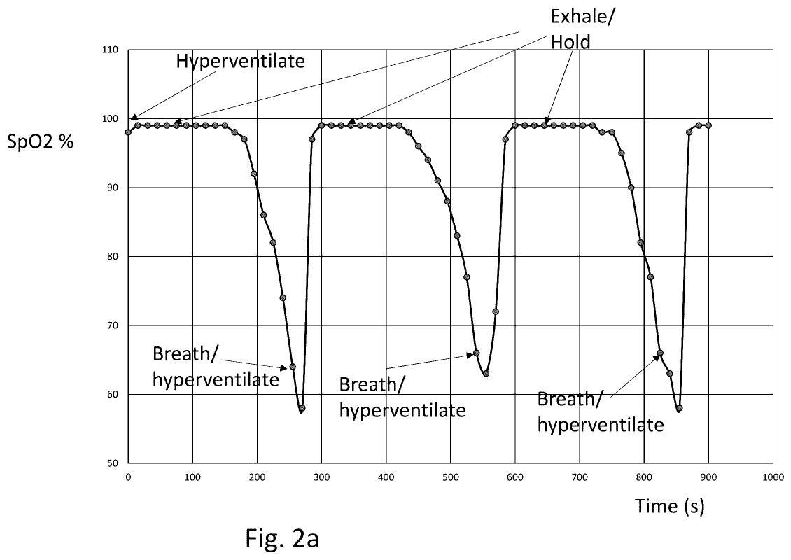

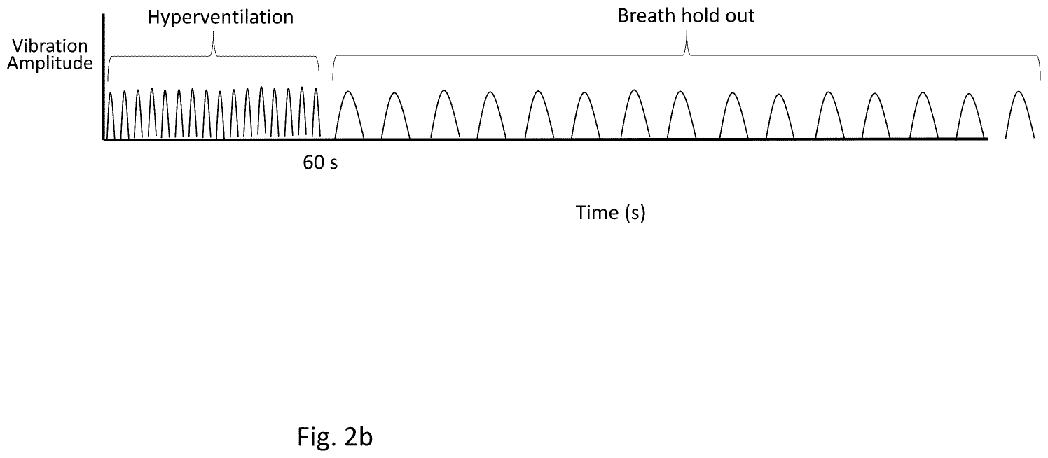

The controller 12 may also be coupled to the SpO2 transducer 52, and programmed to read the oxygen saturation level and to make decisions based on this measured level. The interrelationships of these components and their functioning are described further below with respect to FIGS. 2-8. When steps are described in a particular process, it should be understood that these steps are generally performed by the appropriately programmed microprocessor or controller 12. It should also be understood that these components may be mounted or included in a variety of systems, including a vest worn around the torso, or a massage chair as mentioned above, or any of a number of other exemplary systems.

In one embodiment, for example, the user could be sitting in the massage chair, or the vibrational unit could be worn as a vest, or the user could be standing or sitting on a vibration plate, or many other examples. In addition, the controller 12 messages the user 42 to suggest a variety of breathing types, which could include hyperventilation, hyperventilation over a certain time, hyperventilation over a certain number of breaths, hyperventilation with a certain cadence. The hyperventilation instruction could include 20 to 40 breaths, each with a period of approximately 2-5 seconds. Other controller 12 messages can include suggesting the user stop breathing, exhaust the air from the user's lungs and hold that breath out.

FIG. 1 also shows the blood oxygen saturation sensor 52 which reads the user's blood oxygen saturation value (SpO2) at some position on the user's 42 body. The controller 12 receives the SpO2 value from sensor 52 and uses that value to both control the messages to the user as well as control the vibration device. The controller 12 can then repeat the cycle of hyperventilation and breathe out and hold as many times as desired.

FIG. 2a shows a result of the system of FIG. 1, in this case urging the user to hyperventilate at t=0 until t=60 s, then urging the user to hold breath out beginning at 60 s onward as possible for the user 42. FIG. 2a shows the resulting SpO2 value versus time including an exemplary time-dependent profile of the oxygen saturation level of the user in the therapeutic system; FIG. 2b shows the time-dependent vibration amplitude applied to the user and the associated breathing profile of the user in the therapeutic system.

In this embodiment, the SpO2 sensor 52 is conveniently located on the user's forefinger, however measurements could be made in other areas of the body with similar results. The SpO2 holds near full saturation (close to 100%) for a certain time and then the value falls as oxygen is depleted in the body. The controller 12 may be reporting the SpO2 levels to the user 42, which makes the user's control of breath much easier and more pleasant. The SpO2 sensor 52 may measure a reduction in oxygen saturation level of at least 4%. At some point, the user decides to breathe (which may or may not be from a suggestion from the controller based on time or SpO2 level) and the SpO2 level returns to near saturation. The controller 12 then repeats the process. Alternatively, the user may hold the breath out until a predefined oxygen saturation level is achieved.

FIG. 2b shows the result of the controller 12 driving the vibration device. The vibration device may be any device capable of generating a vibration which is then applied to the user's body 42. The device could be, for example, an array of electromagnetic solenoids, a plurality of unbalanced motors, a piezoelectric actuator and membrane to name just a few. Accordingly, FIG. 2b shows the time-dependent vibration amplitude applied to the user and the associated breathing profile of the user in the therapeutic system.

In this example, FIG. 2b shows a rhythmic envelope of vibrations turning on for a period of time and then turning off. The rhythm of the oscillations could be chosen to match the hyperventilation rhythm during that suggestion time, for example breathing in when the vibrations are on and breathing out when the vibrations are off, and then altered during the exhale and hold time, as shown as an example in FIG. 2b. (Only 1 cycle of hyperventilation and breath hold out is shown in FIG. 2b.) Together, FIG. 1, FIG. 2a and FIG. 2b show a system which will substantially reduce the user's arterial stiffness.

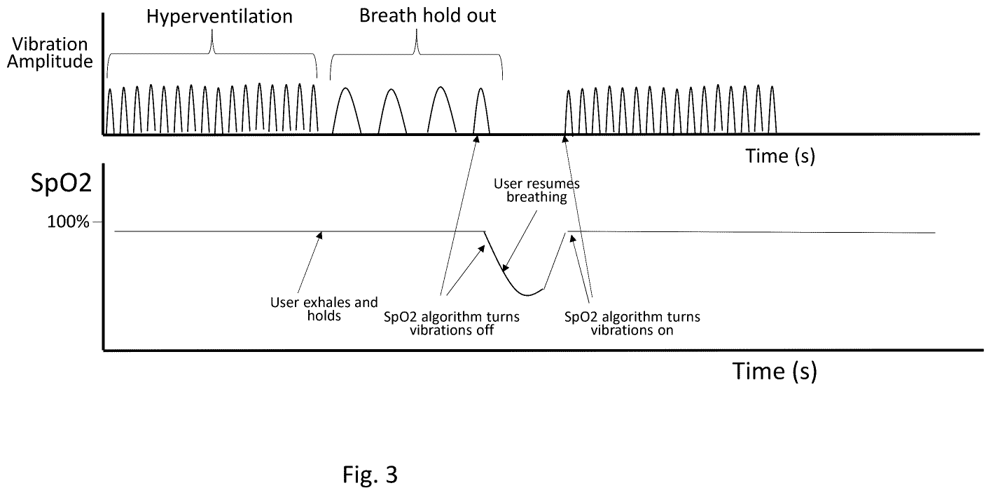

FIG. 3 shows the result of starting with hyperventilation, with an appropriate drive of the vibration device 22 and suggestion from the controller 12. As an example, the Controller could suggest the user breathe in as the vibrations turn on and breathe out as the vibrations turn off. This is followed by a breathe out and hold suggestion from the controller 12, coupled with a change in the vibration timing and amplitude. In this example, when the SpO2 sensor registers the value dropping (sufficiently larger drop than any noise background), the controller 12 changes the vibration device 22 (in this case, amplitude goes to zero). Notification by the controller 12 to the user of the SpO2 level is helpful for the user to control continuing to hold breath out and subsequently when to breathe. When the user 42 resumes breathing, the SpO2 value will increase (after a delay) and the Controller then changes the vibration device 22 to indicate a breathing rhythm for the user and aid hyperventilation.

Many variants can be made for the overall protocol, for example changing the vibration device 22 amplitude and timing for the various stages, changing the levels of SpO2 which trigger vibration device 22 and audio messaging changes. One embodiment is for the controller 12 to instruct the user 42 to hyperventilate at a cadence of a cycle every 2-5 seconds, and drive the vibration with the same rhythm with the vibrations on for a count of a couple of seconds and then vibrations off for a couple of seconds, then repeat. At the suggestion of the breath hold out, the period of the vibrations is increased, typically to a period of 10 to 30 seconds. As the SpO2 value, as measured by the sensor 52, is dropping, the controller 12 stops the vibration device 22 and then resumes to hyperventilation when the SpO2 value begins to climb post-breathing. The use of the SpO2 device also allows safety limits to be placed on the breath-hold phase to instruct the user to breathe as the SpO2 reaches a limit.

In some embodiments, the messaging device may instruct the user to breathe rapidly and inflate and deflate the user's lungs.

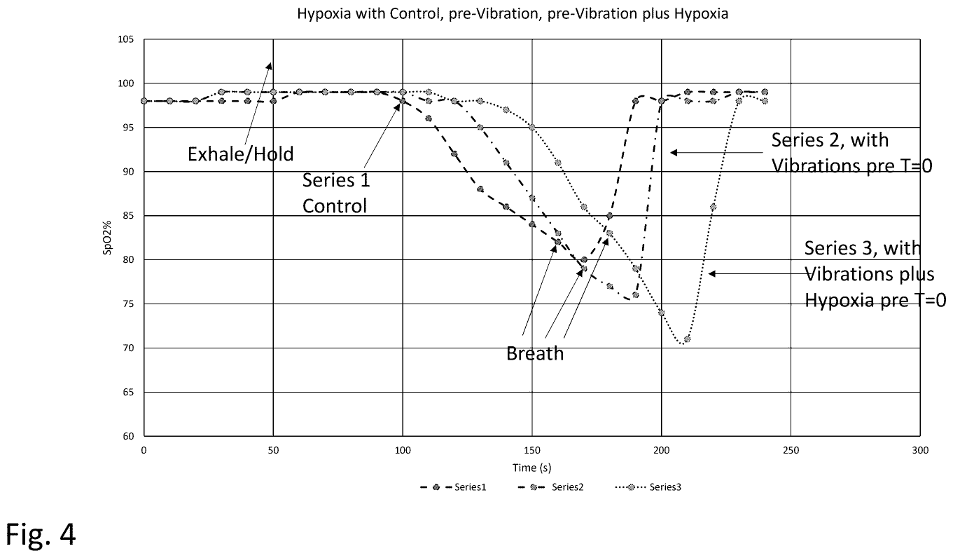

The data in FIG. 4 demonstrate the surprising results of the novel system described here. FIG. 4 shows the SpO2 value of a 65-year-old subject in which the controller 12 has instructed the user 42 to hyperventilate at t=0 until t=60 s, then hold breath out. Series 1 shows a “control” case in which there have been no vibrations applied to the user. The SpO2 naturally falls in time, and at ˜84% saturation, the user is instructed by the Controller to breathe and hyperventilate. Thus the SpO2 recovers to saturation as shown.

FIG. 4 also shows Series 2, in which the user is exposed to the vibration device 22 operating in rhythm of oscillations with a 6 s period, on for 3 seconds and off for 3 seconds, for 10 minutes just prior to commencing the Series 2 data shown. The user has been “pre-conditioned” with the vibrations. Series 2 shows a pronounced change from the “control” condition, with the onset of SpO2 drop happening much later in time.

FIG. 4 also shows Series 3, in which the user 42 is exposed to the same vibration device 22 condition as Series 2, but in the 10 minute pre-conditioning phase, the user is instructed by the controller 12 to hyperventilate and breathe out and hold in cycles, reaching approximately 84% SpO2 in each cycle before returning to saturation. So, in Series 3, the user has been “pre-conditioned” with both the vibrations as well as intermittent hyperventilation and hypoxia. Series 3 shows a remarkable and surprising change in the user's condition, with the onset of SpO2 drop happening about 2 times later than the control case.

In all three Series for FIG. 4, the user's heart rate is closely monitored and was always 65 beats/minute+−3 beats/min. The SpO2 is measured at the user's fingertip, and the user is quiescent during the entire procedure. The explanation for the effect in Series 2 and 3 is that the vibration coupled with the intermittent hypoxia significantly dilates the user's vasculature, delaying the arrival of the oxygen depleted blood to the finger. Also note that the time from breathing to regaining oxygen saturation is much longer in Series 3, again with the explanation that the vascular is dilated and so the velocity of newly oxygenated blood is reduced in that case. It's clear that the combination of both the vibration and the hyperventilation/hypoxia cycling has a profound physiological effect on the user.

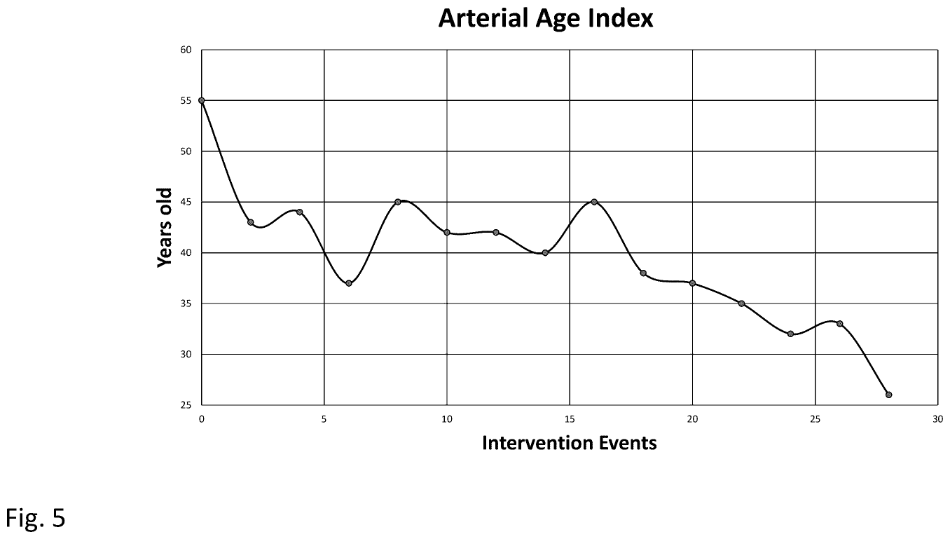

FIG. 5 shows data from a Meridian DPA (digital pulse analyzer) device which measures the pulse plethysmograph of a user's forefinger, averaging over 1 minute of heart beats and can provide a large amount of data on the user's cardiovascular system including parameters correlated to aortic stiffness as well as the user's “Arterial Age Index”. This analysis has been extensively studied, and while the details of the measurements and algorithms is complex, the Arterial Age Index is a useful tool to measure many effects which are occurring as someone ages, including stiffening of the arteries. Studies using the Meridian DPA have been published in the scientific literature and correlated to arterial stiffness parameters such as aortic stiffness in particular. FIG. 5 shows a remarkable and surprising drop in the Arterial Age Index from 55 to 26 years old, as a result of using the current invention protocol and control and measurement system. The x-axis corresponds to the “intervention events” which are uses of the system of FIG. 3 for roughly 1 hour per day over a period of a month. This is a profound result.

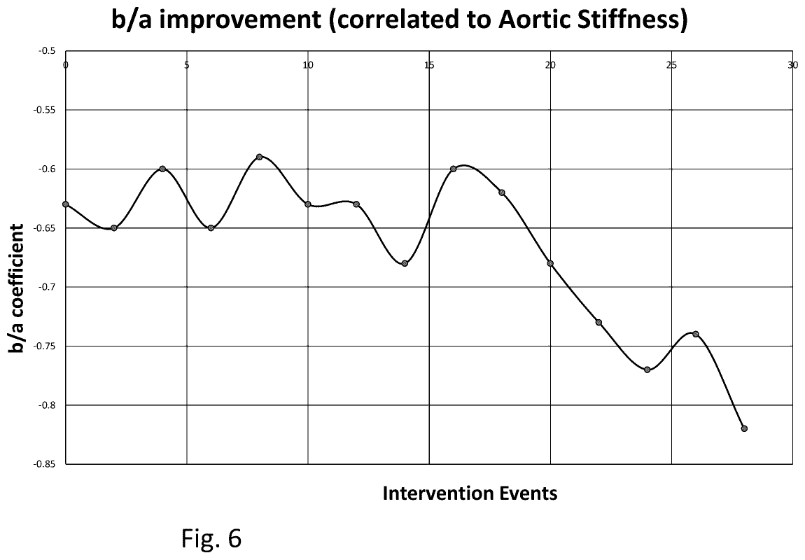

FIG. 6 shows additional data from the Meridian DPA device measured concurrently with the Arterial Age Index data of FIG. 5. FIG. 6 shows the “b/a” coefficient which is the ratio of the first two Second-Derivative peaks during the heart beat cycle as the pressure pulse arrives at the forefinger where the measurement is taken. As established in the literature, the b/a coefficient is well correlated to the aortic stiffness, and so the decrease in this value with usage in the current invention protocol is very significant. No such drop in aortic stiffness has been reported in the literature.

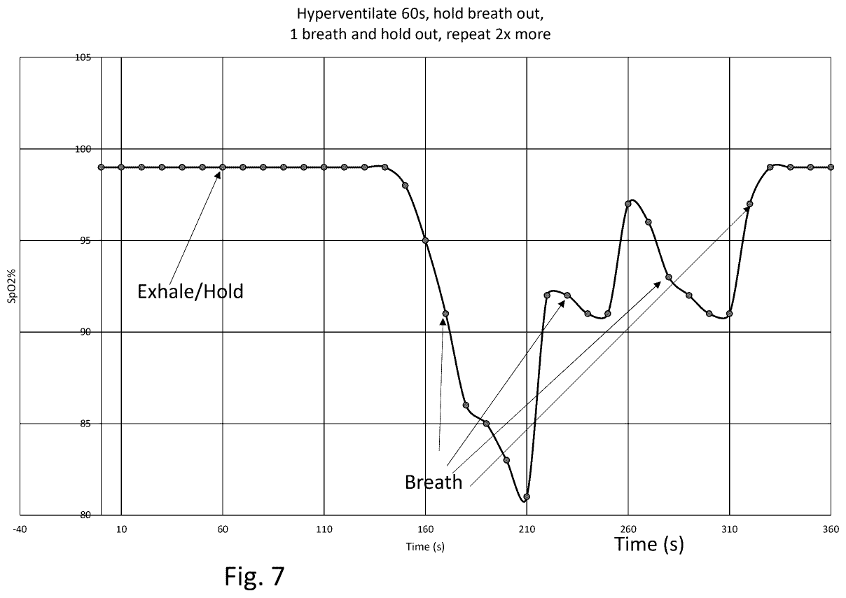

FIG. 7 shows the more complex blood oxygenation saturation level associated with a more complex breathing pattern. The controller 12 instructs the user to breathe for 60 s, then exhale and hold breath out. The controller 12 issues SpO2 values to keep the user 42 informed as the SpO2 is dropping. As the SpO2 goes below a pre-set value, the controller 12 issues an instruction for the user to breathe in once only, then breathe out and hold. The SpO2 value will continue to decrease for a time but then increase (as a result of the one breath), and the controller 12 can issue a repeated instruction to breathe in once only, then breathe out and hold. This can be repeated until the user decides to breathe continuously, bringing the SpO2 back above a preset level. The controller 12 can then restart the cycle with breathing for 60 s. A pattern such as the one shown in FIG. 7 is an efficient way for the user to be continuously changing the SpO2 value in the bloodstream in different parts of the body while maintaining an hypoxic state. When coupled with the vibrational modes driven by the controller 12 (for example in the style of FIG. 3 modified for the 1 breath only mode), the mode shown in FIG. 7 is an option for repeating the vibration enhanced intermittent hypoxia.

Another method for extending the intermittent hypoxia is, after the breath out and hold step, and upon initiating a breath, the controller instructs the user to breathe in a shallow manner. This can extend the hypoxia in the 70-90% range as measured by SpO2 on a finger, for example, to many minutes. Then the controller could issue the instruction to breathe normally or to hyperventilate as has been described.

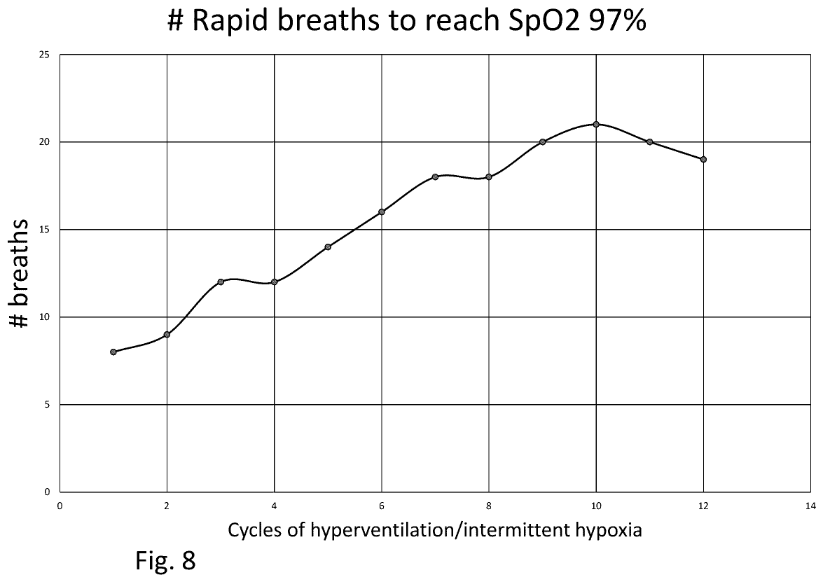

FIG. 8 shows the number of rapid breaths taken to restore the blood oxygenation saturation level to >97% after a hyperventilation/breath hold out cycle, as a function of the number of cycles the user performs with the system. Note that generally the number of rapid breaths must increase after more cycles, presumably due to the increased diameter of the vasculature in the extremities. Because of this effect, it is preferable to have the Controller use this information, and instruct the user to continue to hyperventilate for a certain time (or a certain number of breaths) after it detects the SpO2 exceeds a certain value (e.g. 97%).

The disclosed system may be regarded as both novel and significant, as the reduction in arterial stiffness and resulting improvement in effective arterial age is far beyond anything reported and material in human cardiovascular disease. And while the biology and biochemistry of humans is extremely complex with a large number of unknowns, herein we will nevertheless endeavor to offer a plausible and compelling mechanism of action for the system.

The remodeling of tissue including the change in stiffness of arteries, reduction in atherosclerosis, and angiogenesis is possible and in the literature, nitric oxide (NO) is a significant candidate. There are published scientific papers which lay out the promise of NO for remodeling the cardiovascular system, and yet finding a solution for humans has, until the current invention, proven elusive.

In the current invention, NO is generated from the vibration device. The underlying mechanism for the significant increase in blood flow following vibration may be due to pulsatile endothelial stress resulting in increased endothelial nitric oxide, synthase activity, and NO concentration which in turn dilates the arteries.

But a conundrum is that vibration by itself, even though it generates NO within the human body, does not remodel the cardiovascular system to any degree of what is shown herein. It is the combination with intermittent hypoxia which is both novel and fruitful, as shown in FIG. 4, FIG. 5, and FIG. 6. The mechanism for this effect is that a substantial fraction of NO is carried in the bloodstream as a thiol-compound, S-nitrosothiol (SNO), in turn bound to hemoglobin within the red blood cells. The SNO is released as the hemoglobin becomes de-oxygenated which leads to dilation of the blood vessels. Thus, the NO, already increased by the vibration, is maximally released in the vasculature as a result of the hypoxic conditions, consequently driving enhanced vasculature dilation and also promoting remodeling of the tissue. Cycling this intermittent hypoxia in the presence of vibration (to greatly enhance the available NO) is central to this invention.

The hyperventilation part of the sequence in the current invention serves to deplete the carbon dioxide (CO2) from the bloodstream before the intermittent hypoxic breath-out-and-hold sequence. The drive to breathe in humans is mostly driven by the CO2 level, and so depleting the CO2 allows the user to more comfortably hold breath out to achieve the significant drop in SpO2 reported herein. Furthermore, and importantly, the vibration-induced nitric oxide in the present invention allows the user to much more easily reach low SpO2 levels.

Another effect from vibration enhanced intermittent hypoxia is that many individuals may experience optical hallucinations. These hallucinations can be helpful for the user to gauge their performance, for example comparing their hallucinations to prior uses of the device or comparing over time in the same use. To best witness the hallucinations, a light-blocking mask can be worn by the user to cover the eyes, so that any brightness detected in the eye is not a result of any actual light. One mode of hallucinations is that a change in light level can be perceived as the SpO2 level drops, and after one or more breaths, a bright field or fractional field is witnessed with a delay from the breath. The delay is an indicator of the time for oxygenated blood to arrive at the eyes from the lungs and in turn is a function of the arterial diameters leading to the eyes and the user's cerebral blood pressure. Multiple colors can be seen as well as multiple patterns, providing the user with a useful reference.

For some users, the procedures described herein can potentially lead to dangerously low levels of the user's SpO2. For safety reasons, the controller 12 is programmed to issue a command, through audio or display, and which may be accompanied by a sharp sequence of vibrations such as a series of short vibrational bursts, for the user to breathe. This is very important, as uncontrolled hypoxia can lead to morbidity issues as experienced, for example, by individuals with sleep-disordered breathing problems.

Other ways of ensuring the efficacy and safety of the users 42 may include at least one of the following: instructing the user to perform at least one of the following: self-check to ensure the user has continued to hold breath out, hum to ensure the user has continued to hold breath out; and breathe when the blood oxygen saturation is below a predefined level.

Other embodiments of this system may include additional components which may enhance the user's experience. Such additional components may include a blindfold to block extraneous light, and nostril pincers that reduce or impede the flow of air.

Accordingly, a system is described which uses intermittent periods of self-induced hypoxia, in conjunction with a repetitive series of vibrations to improve the aortic elasticity, which is a primary defining of age-correlated hypertension. The therapeutic device for controlling breath of a user, may include a controller, a vibration device, a messaging device; an oxygen saturation sensor which measures an oxygen saturation level in the blood, and wherein the controller is programmed to direct the vibration device to create a vibration and apply it to the user, while instructing the user through the messaging device to exhale the breath and hold the breath out and wherein the oxygen saturation sensor reports the oxygen saturation level to the user through the messaging device.

Within the device, the messaging device may be an auditory or verbal signal. The controller may be programmed to control a vibration amplitude as a function of time, to measure the oxygen saturation level of the user, and to adjust the vibration amplitude based on the oxygen saturation level. The messaging device may instruct the user to breathe rapidly and inflate and deflate the user's lungs. The controller may be programmed to instruct the user to then breathe out and hold breath out. The controller may be further programmed to instruct the user to continue to hold breath out as a repeated reminder for encouragement until an oxygen saturation level has reached a pre-set value. The controller may be further programmed to instruct the user to perform at least one of the following: self-check to ensure the user has continued to hold breath out, hum to ensure the user has continued to hold breath out; and breathe when the blood oxygen saturation is below a predefined level.

The controller may be further programmed to instruct the user to breathe when the blood oxygen saturation is below a predefined level. The controller may be further programmed to instruct the user to breathe once when the SpO2 falls below a set level and hold breath out. The controller may be further programmed to instruct the user to breathe in a shallow manner. The controller may also be programmed to produce a record of the blood oxygenation saturation level over time during the use of the device. The controller may further be programmed to adjust the instruction for hyperventilation as a result of the measurement of the blood oxygen saturation level.

The therapeutic device may also include a mask which covers the eyes of the user. The therapeutic device may further include a clamp which squeezes nostrils of the user, reducing air flow to lungs via the nostrils, or a plug which blocks air flow into the nostrils. The therapeutic device may also include a blood pressure monitor which measures the user's blood pressure. The therapeutic device may operate at an average vibrational power of at least 30 watts.

The amplitude of vibrations may increase and decrease in a period based on breathing.

The controller may be programmed to reduce the amplitude of vibrations, including to zero amplitude, as the user is instructed to breathe out and hold out. The controller may command the vibration device to operate for a significant time, greater than 2 minutes, and then the controller issues a message delivered to the user which includes instruction to first hyperventilate and then breathe out and hold breath out.

Also disclosed herein is a method for controlling the breathing of a user. The therapeutic method may include providing a controller coupled to a vibration device, a massaging device and an oxygen saturation sensor, controlling the vibration device to create a vibration and apply it to the body of the user; instructing the user to exhale the breath and hold the breath out; reporting the oxygen saturation level to the user through the messaging device; and repeating these steps until a predefined oxygen saturation level is achieved.

Within the therapeutic method, the messaging device may be an auditory or verbal signal. Within the therapeutic method, the controller may control a vibration amplitude as a function of time, to measure the oxygen saturation level of the user, and to adjust the vibration amplitude based on the oxygen saturation level. The therapeutic method may further include instructing the user to breathe rapidly, and inflate and deflate the user's lungs. The therapeutic method may further include instructing the user to then breathe out and hold breath out. The therapeutic method may further include instructing the user to continue to hold breath out as a repeated reminder for encouragement until an oxygen saturation level has reached a pre-set value.

Within the therapeutic method, the messaging device may be an auditory or verbal signal. Within the therapeutic method, the controller may control a vibration amplitude as a function of time, to measure the oxygen saturation level of the user, and to adjust the vibration amplitude based on the oxygen saturation level. The therapeutic method may further include instructing the user to breathe rapidly, and inflate and deflate the user's lungs. The therapeutic method may further include instructing the user to then breathe out and hold breath out. The therapeutic method may further include instructing the user to continue to hold breath out as a repeated reminder for encouragement until an oxygen saturation level has reached a pre-set value.

The therapeutic method may further include instructing the user to perform at least one of the following: self-check to ensure the user has continued to hold breath out, hum to ensure the user has continued to hold breath out, and breathe when the blood oxygen saturation is below a predefined level. The therapeutic method may further include instructing the user to breathe when the blood oxygen saturation is below a predefined level. The therapeutic method may further include instructing the user to breathe once when the SpO2 falls below a set level and hold breath out. The therapeutic method may further instruct the user to breathe in a shallow manner.

The therapeutic method may further include providing a mask which covers the eyes of the user. The therapeutic method may further include providing a clamp which squeezes nostrils of the user, reducing air flow to lungs via the nostrils, or a plug which blocks air flow into the nostrils. The therapeutic method may further include increasing or decreasing the amplitude of vibrations in a period based on breathing. The therapeutic method may further include reducing the amplitude of vibrations, including to zero amplitude, as the user while instructing the user to breathe out and hold out. The therapeutic method may further include commanding the vibration device to operate for a significant time, greater than 2 minutes, and then instructing the user to first hyperventilate and then breathe out and hold breath out. The therapeutic method may further include providing a blood pressure monitor which measures the user's blood pressure. The therapeutic method may further include producing a record of the blood oxygenation saturation level over time during the use of the device. The therapeutic method may further include instructing the user to hyperventilate based on the measurement of the blood oxygen saturation level. The therapeutic method may further comprise applying an average vibrational power of at least 30 watts.

While various details have been described in conjunction with the exemplary implementations outlined above, various alternatives, modifications, variations, improvements, and/or substantial equivalents, whether known or that are or may be presently unforeseen, may become apparent upon reviewing the foregoing disclosure. Accordingly, the exemplary implementations set forth above are intended to be illustrative, not limiting.

What is claimed is:

1. A therapeutic method for controlling breath of a user, comprising:
providing a controller, a vibration device, a messaging device and an oxygen saturation sensor;
measuring an oxygen saturation level with the oxygen saturation sensor;
controlling the vibration device with the controller to create a vibration and apply it to the user;
instructing the user to exhale the breath and hold the breath out;
reporting an oxygen saturation level from the oxygen saturation sensor, to the user through the messaging device until at least a 4% reduction in oxygen saturation level is achieved; and repeating these steps.

2. The therapeutic method of claim 1, wherein the messaging device is an auditory or verbal signal.

3. The therapeutic method of claim 1, wherein the controller is programmed to control a vibration amplitude as a function of time, to measure the oxygen saturation level of the user, and to adjust the vibration amplitude based on the oxygen saturation level.

4. The therapeutic method of claim 1, further comprising:
instructing the user to breathe rapidly, and inflate and deflate the user's lungs.

5. The therapeutic method of claim 4, further comprising instructing the user to then breathe out and hold breath out.

6. The therapeutic method of claim 5, further comprising:
instructing the user to continue to hold breath out as a repeated reminder until an oxygen saturation level has reached a pre-set value.

7. The therapeutic method of claim 5, further comprising:
instructing the user to perform at least one of the following: self-check to ensure the user has continued to hold breath out, hum to ensure the user has continued to hold breath out, and breathe when the oxygen saturation level is below a predefined level.

8. The therapeutic method of claim 5, further comprising:
instructing the user to breathe when the oxygen saturation level is below a predefined level.

9. The therapeutic method of claim 5, further comprising:
instructing the user to breathe once when the oxygen saturation level falls below a set level and hold breath out.

10. The therapeutic method of claim 5, further comprising:
instructing the user to breathe in a shallow manner.

11. The therapeutic method of claim 1, further comprising:
providing a mask which covers the eyes of the user.

12. The therapeutic method of claim 1, further comprising:
providing a clamp which squeezes nostrils of the user, reducing air flow to lungs via the nostrils, or a plug which blocks air flow into the nostrils.

13. The therapeutic method of claim 5, further comprising increasing or decreasing the amplitude of vibrations in a period based on breathing.

14. The therapeutic method of claim 5, further comprising:
reducing the amplitude of vibrations, including to zero amplitude, as the user while instructing the user to breathe out and hold out.

15. The therapeutic device of claim 1 further comprising:
commanding the vibration device to operate for a significant time, greater than 2 minutes, and then instructing the user to first hyperventilate and then breathe out and hold breath out.

16. The therapeutic method of claim 1, further comprising:
providing a blood pressure monitor which measures the user's blood pressure.

17. The therapeutic method of claim 1, further comprising:
producing a record of the oxygenation saturation level over time during the use of the device.

18. The therapeutic device of claim 5, further comprising instructing the user to hyperventilate based on the measurement of the oxygen saturation level.

19. The therapeutic method of claim 1, further comprising: applying an average vibrational power of at least 30 watts.
